(19) United States
(12) Patent Application Publication
(10) Pub. No.: US 2007/0093877 A1
(43) Pub. Date: Apr. 26, 2007

(54) System for maintaining normal health of retinal cells and promoting regeneration of retinal cells

(76) Inventors: Michael C. Beecham, Las Vegas, NV (US); James E. Beecham, Las Vegas, NV (US)

Correspondence Address: Robert J. Sayfie, Suite 200, 42717 Woodward Avenue, Bloomfield Hills, MI, 48304, US

(21) Appl. No.: 11/447,697

(22) Filed: Jun. 6, 2006

Related U.S. Application Data
(60) Provisional application No. 60/730,262, filed on Oct. 26, 2005.

(51) Int. Cl. A61N 1/00

(52) U.S. Cl. 607/53

(57) ABSTRACT

Disclosed is a system for maintaining normal health of photoreceptor cells and promoting regeneration of photoreceptor cells, and methods thereof. The system comprises: an implant capable of being implanted in a region in proximity to retina of an eye; and an electrical stimulation unit disposed in a region around the eye. The electrical stimulation unit induces an electrical current in the implant for electrical stimulation of the retina of the eye, thereby maintaining normal health of photoreceptor cells and promoting regeneration of photoreceptor cells in the retina.

### CROSS REFERENCE TO RELATED APPLICATION

This application claims priority pursuant to 35 U.S.C. §119(e) to U.S. Provisional Application No. 60/730,262, filed Oct. 26, 2005, which application is specifically incorporated herein, in its entirety, by reference.

### FIELD OF THE INVENTION

The present invention relates generally to medical treatment of ocular diseases, and, more particularly, to systems and associated methods for maintaining normal health of retinal cells (e.g., photoreceptor cells) and promoting regeneration of retinal cells by electrical stimulation for therapeutic purposes.

### BACKGROUND OF THE INVENTION

Developments in the area of science and technology have contributed tremendously to the advancement of medical science. The technological developments have not only helped scientific teams in understanding medical disorders, but, have also contributed immensely in providing various solutions including systems and methods for addressing such disorders.

The eye forms an important organ of the human body and, as such, disorders of the human eye have always posed challenges to the scientific community. Blindness is one such disorder that can be attributed, in some cases, to the loss of photoreceptor cells (rods and cones) caused by defects in the retina, retinal pigment epithelium, choroids, and other as yet unknown causes. Retinal degenerative diseases cause the loss of function of a select population of photoreceptor cells. One such progressive disorder is macular degeneration. More specifically, in macular degeneration, the retinal cells, i.e., photoreceptor cells degenerate, while other cells in the eye such as retinal nerve cells are maintained. This is not a one time sudden disorder leading to an immediate complete loss of the photoreceptor cells, thereby leading to complete sudden blindness. Rather, the disorder, macular degeneration, causes a continuous progressive degeneration of the photoreceptor cell function, particularly in an older person, causing a slow loss of central visual acuity by the patient and finally a nearly complete loss of central sight, termed as blindness, in particular in the central area of vision, macula of the eye. The loss of photoreceptors cells leads to the inability of the retina to receive and convert light and in turn send normal signals to the retinal nerve cells. The final stage of macular degeneration is when the photoreceptors cells at the macula are diseased or dead. However, in some cases of macular degeneration, the retinal nerve cells may be viable and functioning alive when other retinal cells such as photoreceptors cells are dead or malfunctioning. As such, several approaches are available providing means of treating macular degeneration at the late or end stage, such as, transplanting a computer chip device capable of receiving light, and then projecting an image onto retinal nerve cells of a blind patient, thereby enabling such patients to overcome natural blindness by providing computerized vision.

A preferable method for handling macular degeneration would be to treat the disease early in the course and delay the progression of the disease, thereby, delaying photoreceptor cell degeneration and delaying blindness. In this manner, any final implantation of a computerized chip with pixel elements for overcoming blindness would be delayed or possibly avoided. One such approach to delay the progression is the electrical simulation of retinal cells and photoreceptor cells and increasing blood supply for the purposes of delaying the macular degeneration and regenerating such photoreceptor cells and/or returning their normal function.

Developments have been seen in the field of application of electric current to the eye for treating ocular diseases. The prior art related to such development include the following:

U.S. Pat. No. 5,522,864 discloses steps of: placing a positive electrode of a direct current source in electrical contact with a closed eyelid of a subject suffering from macular degeneration and other ocular pathology; placing a negative electrode of the source in electrical contact with the posterior neck of the subject; and causing a constant direct current of 200 microampere to flow between the electrodes through the subject for about 10 minutes. The source can be a portable, battery powered constant direct current generator that is affixed to the subject. The patent involves a complex electrical circuitry and mandates the user to wear one electrode over the closed eyelid of the affected eye and does not completely improve vision or provide any ocular implant that can permanently cure the disease.

U.S. Patent Publication Number 20030233137 discloses a trans-cutaneous electrical nerve stimulation device and method using a micro current with a carrier signal and a square wave form for promoting cell repair and/or healing for example in macular degeneration. This method and nerve stimulation device is packaged to require no input from a user and a user must only apply the electrodes to the correct part of the body and start the preprogrammed sequence of electrical currents. The method involves applying bursts of direct current at higher frequencies for shorter periods of time followed by lower frequency bursts of electrical current for longer periods of time. The nerve stimulation device of the publication has a complex configuration and necessitates the user to apply the electrodes to specific prescribed places of the user as prescribed by a health care officer. However, the publication does not provide any means of permanent cure, and does not focus treatment at the macula. The above means are less than completely successful.

Accordingly, there remains a need for systems and methods for ocular disease treatment, and more particularly, retinal, disease treatment in a manner that is safe, fast, easy, convenient, effective and inexpensive.

### SUMMARY OF THE INVENTION

In view of the foregoing disadvantages inherent in the prior art, the general purpose of the present invention is to provide a system for maintaining normal health of retinal cells and promoting regeneration and function of retinal cells and methods thereof configured to include all the advantages of the prior art and other advantages, and to overcome the drawbacks of the prior art. In particular, the present invention provides means to focus electrical micro-current to a precise location of the center of visual acuity, macula of the eye and to the blood supply of the retina. The focused electrical micro-current may lead to increased blood supply to the macula. Also, the present invention provides means to alter micro-chemistry in a focused area of the retina, such as, a scleral region adjacent to the macula to improve nutrition of retinal cells, for example, to aid pigment epithelial cell intake of riboflavin by increasing extracellular calcium content.

In one aspect, the present invention provides a system for maintaining normal health of retinal cells and promoting regeneration of retinal cells. The system comprises: an implant capable of being implanted in a region in proximity to retina of an eye; and an electrical stimulation unit disposed in a region around the eye. The electrical stimulation unit induces an electrical current in the implant for electrical stimulation of the retina of the eye, thereby maintaining normal health of retinal cells and promoting regeneration of retinal cells. The electrical stimulation of the tissue causes blood vessel dilatation, increased nourishment of retinal epithelial cells, and the like.

In another aspect, the present invention provides a method for maintaining normal health of photoreceptor cells and promoting regeneration of photoreceptor cells. The method comprises: disposing an implant having an anchor into a hollow needle; inserting the hollow needle into a sub-retinal region of an eye in a first direction; reversing the needle out of the eye in a second direction, such that, the anchor is embedded in the sub-retinal region, leaving the implant anchored in place in the sub-retinal region; and disposing an electrical stimulation unit in a region around the eye. The electrical stimulation unit induces an electrical current in the implant for electrical stimulation of the retina of the eye, thereby maintaining normal health of photoreceptor cells and promoting regeneration of photoreceptor cells in the retina.

In yet another aspect, the present invention provides another method for maintaining normal health of photoreceptor cells and promoting regeneration of photoreceptor cells. The method comprises: exposing a posterior surface of sclera at posterior of an eye; incising an outer layer of the sclera to form an incision; introducing an implant into the incision, such that, the implant is implanted inside the sclera; disposing an electrical stimulation unit in a region around the eye. The electrical stimulation unit induces an electrical current in the implant for electrical stimulation of the retina of the eye, thereby maintaining normal health of photoreceptor cells and promoting regeneration of photoreceptor cells in the retina.

In yet another aspect, the present invention provides another method for maintaining normal health of photoreceptor cells and promoting regeneration of photoreceptor cells. The method comprises: exposing a posterior surface of sclera at posterior of an eye; disposing an implant with an induction coil and a plurality of micro-needles on an outer layer of the sclera such that, the microneedles of the implant are implanted inside the sclera; and disposing an electrical stimulation unit in a region around the eye. The electrical stimulation unit may optionally be linked to a miniaturized battery disposed within the implant and controlled by a microcircuit. This method induces an electrical current in the implant for electrical stimulation of the retina of the eye via the micro-needles, thereby maintaining normal health of photoreceptor cells and promoting regeneration of photoreceptor cells.

These together with other aspects of the present invention, along with the various features of novelty that characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there are illustrated exemplary embodiments of the present invention.

### BRIEF DESCRIPTION OF THE DRAWINGS

The advantages and features of the present invention will become better understood with reference to the following more detailed description and claims taken in conjunction with the accompanying drawings, wherein like elements are identified with like symbols, and in which:

FIG. 1 is a cross sectional view through an eye 100 with an implant 200 in a sub-retinal region around a lateral edge of macula, according to an exemplary embodiment of the present invention;

FIG. 2 is a perspective view of implant 200, according to an exemplary embodiment of the present invention;

FIG. 3 is a perspective view of a curved needle 250 enclosing the implant 200, according to an exemplary embodiment of the present invention;

FIG. 4 is a cross sectional view through the eye 100 with an implant 300 inside sclera 106 of the eye 100, according to an exemplary embodiment of the present invention;

FIG. 5 is a perspective view of a needle 350 and syringe 352 system introducing the implant 300 into the sclera 106 of the eye 100, according to an exemplary embodiment of the present invention;

FIG. 6 is a cross sectional view through the eye 100 with an implant 400 in a region at the outer surface of the sclera 106 at the posterior of the eye 100, according to an exemplary embodiment of the present invention;

FIG. 7A is a cut-away top view of the implant 400, according to an exemplary embodiment of the present invention;

FIG. 7B is a bottom view of the implant 400, according to an exemplary embodiment of the present invention;

FIG. 7C is a side view of the implant 400, according to an exemplary embodiment of the present invention;

FIG. 7D is a side view of the implant 400 with micro-battery 410, micro-capacitor 414 and microcircuit 412, according to an exemplary embodiment of the present invention;

FIG. 8 is a side view of a subject wearing an electrical stimulation unit in the form of stereo headphone 500, according to an exemplary embodiment of the present invention; and

FIG. 9 is a side view of an electromagnet 506 illustrating the swiveling movement, according to an exemplary embodiment of the present invention.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however that the present invention is not limited to a particular system for maintaining normal health of photoreceptor cells and promoting regeneration of retinal cells, as shown and described. It is understood that various omissions, substitutions of equivalents are contemplated as circumstances may suggest or render expedient, but is intended to cover the application or implementation without departing from the spirit or scope of the claims of the present invention.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The present invention provides a system for maintaining normal health of photoreceptor cells and promoting health and regeneration of retinal cells (e.g., photoreceptor cells). The system comprises at least one implant and an electrical stimulation unit. The implant is capable of being implanted in a region in proximity to retina of an eye, while the electrical stimulation unit may be disposed in a region around the eye, such that, the electrical stimulation unit induces an electrical current in the implant for electrical stimulation of retina, i.e., electrical stimulation of a retinal tissue or a tissue in proximity to the retina (e.g., scleral tissue) and improving retinal blood supply. The electrical stimulation causes the maintenance of normal health of photoreceptor cells and promotes regeneration of photoreceptor cells (rods and cones) and improves blood supply to the retina. The system provides a fast, easy, convenient, safe and inexpensive method for maintenance of normal health of photoreceptor cells and promotion of regeneration of photoreceptor cells in the retina for patients with retinal disease. Treatment of retinal diseases using the system does not require visits to clinics/hospitals for hours of treatment, and may be comfortably used at home or office without any specialized assistance.

The implant may be implanted in any suitable region of the eye in proximity to retina of the eye, such that, the electrical signals generated by the system reaches more effectively to the retinal tissue and scleral tissue including the blood supply of the pigment epithelial cells and the photoreceptor cells in the retina. Exemplary regions include, but are not limited to, a sub retinal region, a region within sclera of the eye, and a region on an outer layer of the sclera at the posterior of the eye. More specifically, the present invention provides an implant that may be implanted in a region just outside the retina on an anterior side or a posterior side, such that, the electrical signals generated by the electrical stimulation unit may effectively reach the degenerating photoreceptor cells via the implant, thereby preventing further degeneration and aid in maintaining normal health of photoreceptor cells, improving blood supply and promoting regeneration of photoreceptor cells. Also, vascular supply to the retina may be stimulated to dilate electrically by the system. Such a dilatation may prevent further degeneration and also further aid in maintaining normal health of photoreceptor cells.

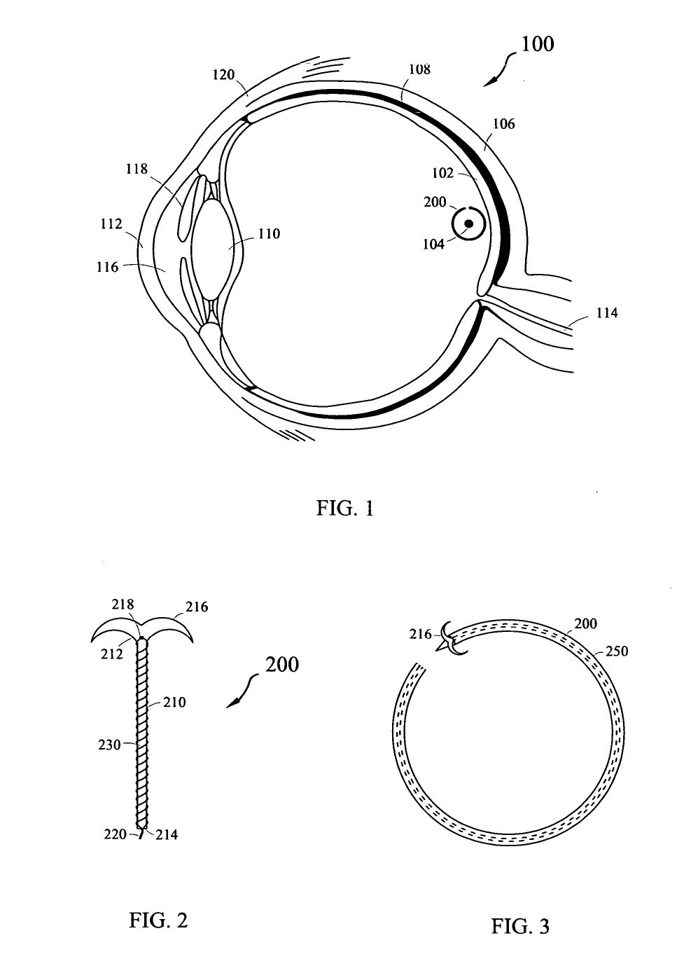

Referring to FIG. 1, in one embodiment, a cross sectional view through an eye 100 with an implant 200 implanted in a sub-retinal region (or alternatively in a sub-choroid region) of the eye 100, is shown. More specifically, the implant 200 can be implanted in the shape of a ring around a lateral edge of macula 104 in the sub-retinal region in proximity to retina 102 of the eye 100. The other components, such as, the sclera 106, choroid 108, lens 110, cornea 112, optic nerve 114, pupil 116, iris 118, and conjunctiva 120 are also represented.

As shown in FIG. 2, the implant 200 is in the form of a flexible suture 210 on which is wound a wire coil 230. The suture 210 has an elongate structure with a first end 212 and a second end 214. The first end 212 of the implant 200 has a fish hook type anchor 216. The implant 200 may be made of a moldable plastic material or any other non-conductive material that provides sufficient flexibility for implanting the implant 200 in a desired manner. Exemplary plastic materials include, but are not limited to, polyolefins, polyesters, polyamides, polyimides, and copolymers and blends thereof.

The wire coil 230 can be engaged to the suture 210 through a hole 218 at the first end 212 of the implant 200. The wire coil 230 may be braided down the elongate structure of the suture 210 from the first end 212 to a position 220 past the second end 214, and then the wire coil 230 can be braided to itself to keep itself in place. The wire coil 230 may be a double wound down structure. The wire coil 230 is made of a conductive material capable of being electrically induced by an electrical stimulation unit. In one embodiment, the wire coil 230 is a copper wire coil.

Referring to FIG. 3, a hollow curved needle 250 enclosing the implant 200, is shown. The curved needle 250 has a gentle curve or up to a radius curve. In one embodiment, the needle 250 has an average internal diameter of about 0.5 millimeter and an external diameter of about 0.75 millimeter, creating a hollow space therein, to suitably enclose the implant 200 with the suture 210 having an average diameter of about 0.2 millimeter and the wire coil having an average diameter of about 0.1 millimeter.

The needle 250 may be used for implanting the implant 200 in the sub-retinal or sub-choroid region of the eye 100. The needle 250 may be inserted into the sub-retinal region in first direction, such as, a counter-clockwise direction around the macula 104. Then the needle 250 can be reversed in a second direction, such as, a clockwise direction out of the eye. The anchor 216 embeds in sub-retinal tissue in the sub-retinal region, keeping the implant 200 in place as the needle 250 is removed out of the eye 100.

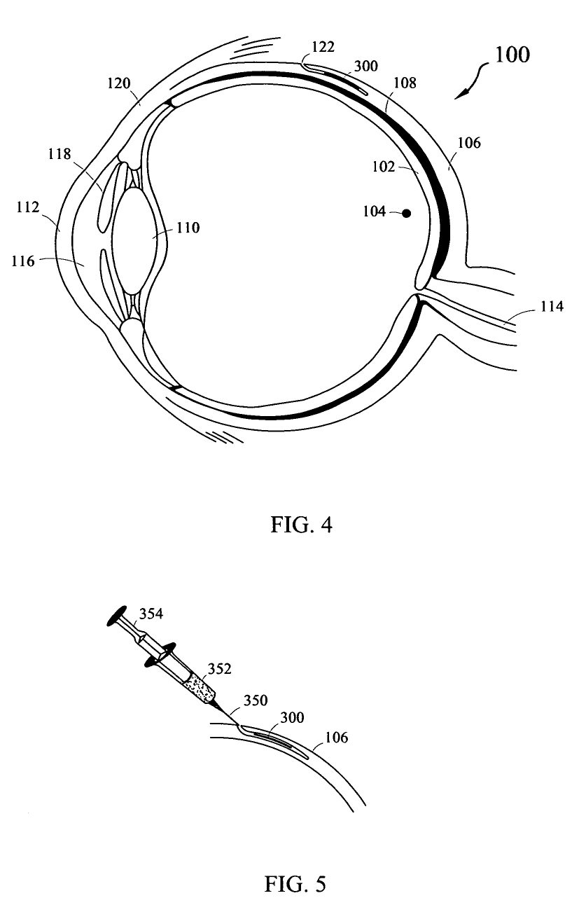

In another embodiment, as shown in FIGS. 4 and 5, an implant 300 (is) implanted in a region within the sclera 106 of the eye 100, is shown. In this embodiment, a posterior surface of the sclera 106 at posterior of the eye 100 is surgically exposed under sterile conditions. Next, a micro-scalpel may be used to incise an outer layer of the sclera 106 to form a pocket-like incision 122. The incision 122 may have a length of about 2 millimeter, a width of about 01.3 millimeter and a depth of about 0.2 millimeter from the outer layer of the sclera 106. The implant 300, as used herein, is in the form of a copper wire coil (e.g., Chaplin® copper wire) having a length of about 1.3 millimeter and an average diameter of about 0.08 millimeter. Optionally, the copper wire may be coiled to have a coil diameter of 0.16 millimeter. The implant 300 with some saline solution/balanced salt solution is placed inside the lumen of a needle 350 (See FIG. 5) or any other needle-like tubular probe that is flexible enough to undergo moderate bending without any blockage or damage to itself. In one exemplary embodiment, the needle 350 may be of about 26-gauge to about 30-gauge in diameter, and more preferably, the needle 350 is 28-gauge in diameter. In one embodiment, the needle 350 is a Microfil® 28-gauge needle.

The needle 350 can be attached to a syringe 352 filled with a suitable quantity of normal saline solution. Next, a front end of the needle 350 may be placed into the front lip of the incision 122 formed by the micro-scalpel. The implant 300 and saline solution can be expelled into the incision 122 by depressing a plunger 354, such that the implant 300 may be disposed in a region within the sclera 106 of the eye 100. The incision 122 can be closed in a sterile manner using a medical adhesive or other common means. The saline solution will reabsorb into the tissue of the sclera 106, while the implant 300 may be implanted in the region within the sclera 106 of the eye 100. The implant 300 may be placed so as to align with blood supply vessel to the macula and cause dilation of said blood inflow.

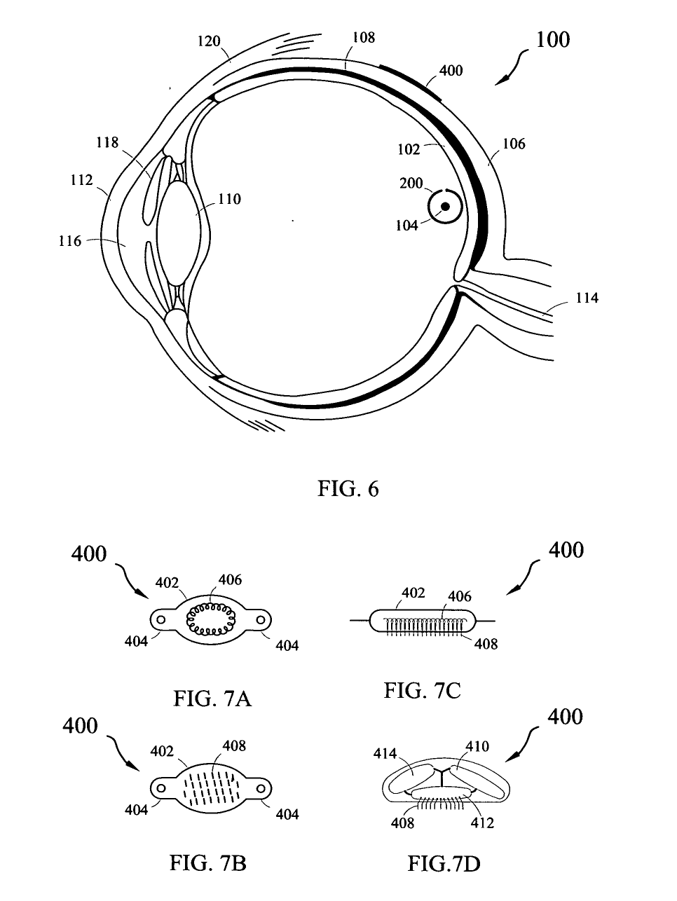

In another embodiment, an implant 400 can be implanted in a region on an outer layer of the sclera 106 at the posterior of the eye 100 (See FIG. 6). The implant 400 is engaged to the outer layer of the sclera 106, the implant having a plurality of microneedles 408 penetrating into the sclera 106 towards the choroid 108 and retina 102. The implant 400 may be implanted using direct pressure, or may be sutured in place. The implant 400 has a structure having an inductive element shielded by an insulation case except where in continuity with the plurality of micro-needles 408 to deliver the electric impulse deeper within the sclera 106, such that, the electrical signals generated by an electrical stimulation unit may effectively reach the scleral tissues, micro-chemistry environment adjacent to the macula, and photoreceptor cells via the implant 400.

For example, as shown in FIGS. 7A-7D, the implant 400 may be a disc-shaped structure 402 with flaps 404. The disc shaped structure 402 has an induction coil 406 on a top surface (FIG. 7A) and the plurality of micro-needles 408 on a bottom surface. The micro-needles 408 conduct the electrical current induced in the implant 400 to the scleral tissue (or choroid tissue) near the macula or blood supply vessels, and, more particularly to, the photoreceptor cells. Alternatively, the implant 400 may contain a micro-battery 410 controlled by a self-contained microcircuit 412 which regulates electrical charge from the micro-battery 410 to be temporarily stored in a micro-capacitor 414. The microcircuit 412 then regulates the time and sequence of discharge of micro-capacitor 414, and thereby causes the stored charge in the micro-capacitor 414 to be discharged into the scleral tissue through the micro-needles 408 (See FIG. 7D).

Also, a plurality of implants may be implanted in and around the sclera, or sub-retinal region, or sub-choroid region, such that, electric signals generated by the electrical stimulation unit may be used to induce electrical current in the plurality of implants, and the plurality of implants in combination induce better blood supply to the critical macular area, better micro-chemistry at the fovea, thereby improving maintenance and regeneration of photoreceptor cells and other associated cells of the retina.

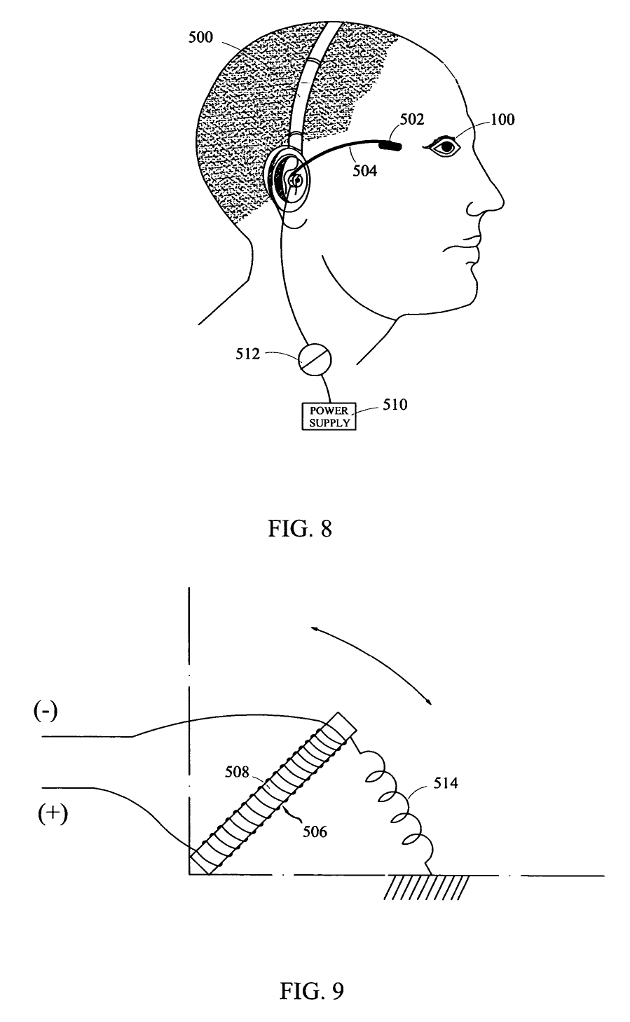

Now, referring to FIGS. 8 and 9, a subject wearing an electrical stimulation unit in the form of stereo headphone 500 with its components there are shown. The headphone 500 can be a standard headphone configured to fit over the ears. The front of the headphone 500 has an extension 502 connected to the headphone 500 through a tubular connection 504 capable of carrying electrical wires and the extension 502 extends to the temple area just besides the eye. The extension 502 may be configured to house an electromagnet 506 on a swivel (not shown) and electrically connected to a servomotor. The electromagnet 506 has an electromagnet core 508 and along with the servomotor are connected to a power supply 510 that provides electric power to the electromagnet 506 and the servomotor in order to power the electromagnet and to turn or swivel the electromagnet to create a moving magnetic field in relation to the implant(s). The power of the electric field produced by the electromagnet can be increased by increasing the voltage via a voltage control 512. In one embodiment, the extension 502 may be in the shape of a cylindrical plastic case of 5 cm diameter and 2 cm thickness. However, configurations of other dimensions are also feasible. The electromagnet 506 within the extension 502 is capable of swiveling 90 degrees instead of rotating 360 degrees and can be accomplished by connecting a spring 514 to one end of the electromagnet core 508 which limits the rotation of the electromagnet 506 to 90 degrees as shown in FIG. 9. The other end of the electromagnet can be electrically connected to a solenoid (not shown). The solenoid pulls the electromagnet 506 about the swivel to cause the 90 degree swivel.

When a subject puts the headphone 500 on, to listen to music, the electromagnet 506 can be activated and the swivel moves the electromagnet 506 to create a moving magnetic field beside each eye. As the extension 502 extends very close to the eye 100 near the temple area, the moving magnetic field created by the electromagnet 506 extends through the retina 102 and may be sufficient to stimulate an electrical current in: the implanted wire coil 230 on the suture 210 in the eye 100; or similarly in the coil 300 in the implant 300 (i.e., the copper wire coil); or in the metallic coil 406 of the implant 400. In case where the suture 210 is non-conductive, the electrical current generated in the wire coil 210 spreads into the sub-retinal tissue. This electrical stimulation of the sub-retinal tissues causes improvement of blood flow to macular tissue and improves local micro-chemistry in order to maintain normal health of photoreceptor cells and promote regeneration of photoreceptor cells.

In an alternative embodiment, the implant 400 may have a battery with a linked computer chip housed within the insulation case. In this embodiment, the implant 400 operates independent of the magnetic field generated by the headphone 500, and instead operates based on an instruction code stored in the computer chip. The battery in this embodiment is electrically connected to the micro-needles 408 and the implant 400 is directed to send micro-current via the micro-needles into the eye 100, according to the instruction code and timer of the computer chip.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is understood that various omissions, substitutions of equivalents are contemplated as circumstance may suggest or render expedient, but is intended to cover the application or implementation without departing from the spirit or scope of the claims of the present invention.

What is claimed is:

1. A system for maintaining normal health of retinal cells and promoting regeneration of retinal cells, comprising:
an implant capable of being implanted through an exterior of an eye into a region in proximity to retina of the eye; and
an electrical stimulation unit disposed in a region around the eye,
wherein the system delivers an electrical current into eye tissue in proximity to the retina of the eye, thereby maintaining normal health of retinal cells and promoting regeneration of retinal cells.

2. The system of claim 1, wherein the implant is implanted in a sub-retinal region around a lateral edge of macula of the eye.

3. The system of claim 2, wherein the retinal cells are photoreceptor cells.

4. The system of claim 1, wherein the implant is in the form of a suture with a wire coil wound around the suture.

5. The system of claim 4, wherein the suture is made of a flexible plastic-like material.

6. The system of claim 5, wherein the suture has an anchor at a first end that embeds in the region in proximity to retina of the eye, keeping the implant in place.

7. The system of claim 1, wherein the implant is a copper wire coil.

8. The system of claim 1, wherein the implant is implanted within sclera of the eye.

9. The system of claim 1, wherein the implant is implanted in a region on an outer layer of sclera at posterior of the eye.

10. The system of claim 9, wherein the implant comprises an induction coil shielded by an insulation case and a plurality of micro-needles penetrating into the sclera.

11. The system of claim 1, wherein the electrical stimulation unit is capable of generating a moving electromagnetic field for inducing the electrical current in the implant.

12. The system of claim 11, wherein the electrical stimulation unit is a headphone having an extension capable of housing an electromagnet on a swivel and a servomotor, the electromagnet and the servomotor electrically connected to a power supply that provides electrical power to the electromagnet and the servomotor through a voltage control.

13. The system of claim 12, wherein a tubular connection carrying electrical wires electrically connects the extension to the headphone.

14. The system of claim 12, wherein the electromagnet is coupled to a spring at one end and to a solenoid at other end.

15. The system of claim 12, wherein upon receiving electrical power from the power supply, the electromagnet gets activated, enabling the swivel to move the electromagnet, thereby creating a moving electro magnetic field.

16. The system of claim 1, wherein the implant comprises a microcircuit linked to a micro-capacitor and linked to a micro-battery.

17. The system of claim 16, wherein the microcircuit coordinates temporary storage of electrical charge in the micro-capacitor, and wherein the microcircuit subsequently coordinates release of the stored electrical charge from the micro-capacitor into the eye tissue in proximity to the retina through a plurality of micro-needles of the implant.

18. A method for maintaining normal health of photoreceptor cells and promoting regeneration of photoreceptor cells, comprising:
disposing an implant having an anchor into a hollow needle;
inserting the hollow needle into a sub-retinal region of an eye in a first direction;
reversing the needle out of the eye in a second direction, such that, the anchor is embedded in the sub-retinal region, leaving the implant anchored in place in the sub-retinal region; and
disposing an electrical stimulation unit in a region around the eye;
wherein the electrical stimulation unit induces an electrical current in the implant for electrical stimulation of retina of the eye, thereby maintaining normal health of photoreceptor cells and promoting regeneration of photoreceptor cells in the retina.

19. The method of claim 18, wherein the electrical stimulation unit is a stereo headphone capable of generating moving electromagnetic field for inducing the electrical current in the implant.

20. A method for maintaining normal health of photoreceptor cells and promoting regeneration of photoreceptor cells, comprising:
exposing a posterior surface of sclera at posterior of an eye;
incising out an outer layer of the sclera to form an incision;
introducing an implant into the incision, such that, the implant is implanted inside the sclera;
disposing an electrical stimulation unit in a region around the eye;
wherein the electrical stimulation unit induces an electrical current in the implant for electrical stimulation of the retina of the eye, thereby maintaining normal health of photoreceptor cells and promoting regeneration of photoreceptor cells in the retina.

21. The method of claim 20, wherein the electrical stimulation unit is a stereo headphone capable of generating moving electromagnetic field for inducing the electrical current in the implant.

22. The method of claim 21, wherein the headphone has an extension capable of housing an electromagnet on a swivel and a servomotor, the electromagnet and the servomotor electrically connected to a power supply that provides electrical power to the electromagnet and the servomotor through a voltage control.
